(19) United States
(12) Patent Application Publication
(10) Pub. No.: US 2012/0244226 A1
(43) Pub. Date: Sep. 27, 2012

(54) TREATMENT COMPOSITION AND METHOD

(75) Inventors: Rodney Wayne Claycomb, Hamilton (NZ); Judith Mary Bragger, Hamilton (NZ)

(73) Assignee: DEC INTERNATIONAL NZ LIMITED, Hamilton (NZ)

(21) Appl. No.: 13/514,489

(22) PCT Filed: Dec. 14, 2010
(86) PCT No.: PCT/NZ2010/000253

§ 371 Date: Jun. 7, 2012

(30) Foreign Application Priority Data
Dec. 15, 2009 (NZ) 582115

(51) Int. Cl. A61K 35/20; A61P 29/00

(52) U.S. Cl. 424/535

(57) ABSTRACT

The present invention relates to a medicament including a treatment composition in combination with at least one anti-inflammatory composition, characterised in that the treatment composition is extracted from whole milk, processed milk or milk derived substance, and wherein the treatment composition contains a cationic fraction which includes two or more components of the whole milk, processed milk or milk derived substance with an isoelectric point of or greater than substantially 6.8.

### TECHNICAL FIELD

This invention relates to a treatment composition and method.

In particular, the present invention relates to a treatment composition and method for use in the prevention or treatment of the systemic or topical effects of inflammation.

### BACKGROUND ART

Humans and animals both suffer from various conditions that can result in the up-regulation of systemic inflammatory pathways. These conditions include cardiovascular disease, osteoarthritis, joint pain and conditions resulting from physical injury.

Inflammation is such a prevalent symptom, that anti-inflammatory drugs make up about half of all analgesics on the market.

Some of the treatments and associated problems are discussed below.

Corticosteroids reduce inflammation and swelling by binding to cortisol receptors. Unfortunately, long-term use of these drugs has severe side effects including amongst other things diabetes, osteoporosis and depression. In addition to concern about side effects, these are synthetic drugs and comparatively expensive leading to consumer resistance.

Non steroidal anti-inflammatory drugs (NSAIDs) alleviate pain by counteracting the COX enzyme. These are a commonly available treatment over the counter and include such examples as aspirin, ibuprofen (commonly sold under the trade mark Nurofen™) and naproxen.

The use of NSAIDs can lead to gastrointestinal effects as well as renal adverse drug reactions. As with the corticosteroids, these drugs are synthetic and there can be consumer resistance to using these.

Consumers have increasingly been looking to using nutraceutical remedies which are derived from natural sources for the anti-inflammatory effects. These can include fruit extracts, curcumin (derived from turmeric), ginger and hyssop. While satisfying the “natural” angle desired by consumers, these products are considerably less effective than their synthetic counterparts.

A popular supplement has been the combination of glucosamine (derived from shellfish) and/or chondroitin (derived from animal cartilage). These have fewer side effects than NSAIDs.

However, while there is some effect on arthritic pain, there seems to be little evidence of slowing of loss of cartilage in the joint.

A trial with 1583 participants in the United States concluded that overall the chondroitin/glucosamine was not significantly better than a placebo.

http://nccam.nih.gov/research/results/gait/qa.htm#c1

http://content.nejm.org/cgi/reprint/354/8/795.pdf

http://rheumatology.oxfordjournals.org/cgi/reprint/43/1/100

It is an object of the present invention to address the foregoing problems or at least to provide the public with a useful choice.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

Throughout this specification, the word “comprise”, or variations thereof such as “comprises” or “comprising”, will be understood to imply the inclusion of a stated element, integer or step, or group of elements integers or steps, but not the exclusion of any other element, integer or step, or group of elements, integers or steps.

Further aspects and advantages of the present invention will become apparent from the ensuing description which is given by way of example only.

### DISCLOSURE OF THE INVENTION

According to one aspect of the present invention there is provided the use of

a treatment composition

in combination with at least one anti-inflammatory composition,

in the manufacture of a medicament for the prevention or treatment of inflammatory activity in an animal,

characterised in that

the treatment composition is extracted from whole milk, processed milk or a milk derived substance,

and the treatment composition contains a cationic fraction which includes two or more components of the whole milk, processed milk or milk derived substance with an isoelectric point of or greater than substantially 6.8.

According to another aspect of the present invention there is provided a method of preventing or treating inflammatory activity in an animal

characterised by the step of

applying to the animal a medicament including a treatment composition extracted from whole milk, processed milk or a milk derived substance, in combination with at least one anti-inflammatory composition,
and wherein the treatment composition contains a cationic fraction which includes two or more components of the whole milk, processed milk or milk derived substance with an isoelectric point of or greater than substantially 6.8.

According to a further aspect of the present invention there is provided a medicament including a treatment composition in combination with at least one anti-inflammatory composition,

characterised in that

the treatment composition is extracted from whole milk, processed milk or a milk derived substance,

and wherein the treatment composition contains a cationic fraction which includes two or more components of the whole milk, processed milk or milk derived substance with an isoelectric point of or greater than substantially 6.8.

Throughout the specification the treatment composition will be referred to as a cationic fraction.

Throughout the specification the term “cationic fraction” shall be taken as meaning a fraction of milk, being cationic components that bind to cation exchange media. The cationic fraction should be taken to include any component of milk which has an isoelectric point of or above substantially 6.8.

Preferably the cationic fraction includes at least three milk derived components. The inventors believe the greater number of components provide a synergistic effect. Plus the manufacturing process to derive the components is easier if more components are extracted in a single step.

The applicant is highly familiar with the properties of such cationic fractions being the subject of various patent applications derived from New Zealand Patent Application No. 547859. In this patent, the cationic fraction is used to prevent or treat bovine mastitis, and infection of cow teats. Although other cationic fractions can be used, the applicant has found that those discussed in this patent work particularly well. One advantage of using a fraction as described in this patent is that the manufacturing process is relatively simple requiring just a single elution of a fraction of bound milk components from a cation exchange material.

A one step elution process decreases the length of extraction time and therefore decreases the possibility of biactives being denatured. It also decreases the time, labour and costs in the extraction process providing a significant advantage especially on a large scale.

Another advantage of using a cationic fraction such as described in New Zealand Patent No. 547859 is that preferred embodiments have a number of immune defence proteins contained therein such as lactoferrin, lactoperoxidase and angiogenin.

The inventors have found a combination of a large number of immune defence proteins found in a cationic fraction acts synergistically together to regulate anti-inflammatory activity.

By way of background lactoferrin on its own has been known to be involved in anti-inflammatory activity. This has even been included with a combination of glucosamine and chondroitin (sold under the trade mark Osteol™) and described in WO 2008/009798. This patent compared the anti-inflammatory activity of two compositions 1) pure lactoferrin (90%) and 2) a composition that included lactoferrin at 45-60% plus other milk proteins such as lactoperoxidase, □-lactoglobulin and lactalbumin. (Neither □-lactoglobulin nor lactalbumin are cationic proteins having isoelectric points of 5.4 and 4.5 respectively). Neither of these compositions showed significant anti-inflammatory activity on their own. When combined with glucosamine and chondroitin sulphate the anti-inflammatory activity of 45-60% lactoferrin (composition 2) was not significantly better than glucosamine and chondroitin sulphate alone. The combination of 90% lactoferrin with glucosamine and chondroitin sulphate showed significantly better anti-inflammatory than glucosamine and chondroitin sulphate alone.

However, the inventors are aware that lactoferrin can actually act as an Alarmin through its role of recruiting neutrophils (www.ncbi.nlm.nih.gov/pubmed/18453607).

While the trend is to extracting individual milk components such as lactoferrin (WO 2009/020481, EP 0753308, and U.S. Pat. No. 6,716,813) there is no mention in the prior art of using a combination of immune defence proteins such as those found in a cationic fraction described above.

As discussed in the Best Modes Section the applicants have found that the use of lactoferrin in particular on its own can cause an inflammatory response. However, the applicants found that the combination of a cationic fraction as described with a known anti-inflammatory provides a highly synergistic effect increasing the anti-inflammatory effects over the use of components individually.

An anti-inflammatory agent is any drug or composition that blocks or inhibits the cascade of inflammatory mediators released in response to injury, irritation or infection. Such an agent might act at the beginning of the cascade, for example proteins produced by recombinant DNA technology to specifically bind to the pro-inflammatory cytokines TNF-alpha or IL-1, or may act further along the cascade, for example steroids which inhibit phospholipase A2, or non-steroidal analgesics (NSAIDs) which inhibit cyclooxygenase (COX) enzymes.

However, in preferred embodiments the anti-inflammatory composition is one that is perceived as “natural” in order to satisfy the public's desire for more “natural” treatments. With the cationic fraction being derived from milk, this is also perceived as a “natural” product, thus making the combination more appealing to the consumer.

Further, the use of the present invention with naturally derived anti-inflammatory agents such as chondroitin and glucosamine has significantly increased their effectiveness. This addresses one of the problems associated with the prior art is that although the more natural products have less side effects, they are also significantly less effective.

It should be appreciated however that if the anti-inflammatory agent used is an NSAID or corticosteroid, the synergistic effect may be useful in reducing the amount of the more powerful drugs required and hence reducing the side effects.

It is believed the mechanism by which the present invention works is as follows:

The cationic proteins in milk include a number of proteins that are part of the innate defense system. The innate defense system is present throughout the body at all times and has non-specific antimicrobial activity that may eliminate small numbers of invading microorganisms without an acute inflammatory response. It is produced at high levels in nasal and tracheal passages, in gastric, genital and opthalmic secretions as well as in the mammary gland. For example, Smolenski et al. (2007) have identified 95 minor milk proteins (other than caseins, β-lactoglobulin and α-lactalbumin), 24 of them associated with host defense. Lactoferrin is the most abundant and has anti-inflammatory activity by binding lipopolysaccharide, but interestingly has also been shown to be pro-inflammatory on its own. Lactoperoxidase protects against free radical damage by taking up peroxide (free radicals) to generate the biocidal compound hypothiocyanite, and is thus both anti-microbial and anti-oxidant. Other proteins of the innate defense system include CLP-1, β-defensin, ribonuclease, and amyloid proteins.

The inventors believe that the reason that the present invention works so well is that the innate defense proteins are both anti-microbial and immunomodulatory, so that a minor invasion of foreign organisms or particles does not elicit an acute inflammatory response. The lactoferrin acts initially to bind and neutralise lipopolysaccharide but also as an Alarmin to recruit neutrophils. However lactoferrin's effect as an Alarmin is modified by the inclusion of the other immune defence proteins within the cationic fraction. Once the neutrophils have been recruited by the lactoferrin, then the combination of the immune defence proteins and the other anti-inflammatory agent can have a greater effect than if the anti-inflammatory agent did not have the benefit of the neutrophil's recruitment.

The medicament may include additional components to that provided by the cationic fraction and the conventional anti-inflammatory. For example, there may be provided a oxygen or peroxide generation system as this can enhance the activity of the proteins within the cationic fraction.

It may also include a cell lysing agent which has the effect of enhancing the anti-microbial activity. This is due to certain organisms having greater resistance if their cell walls are intact.

In other embodiments, there may be included a CLP-1 protein which has the effect of lysing the cell walls of gram positive organisms and thereby enhancing the antimicrobial activity.

It is noted that the present invention can be applied to the treatment subject (which may be human or non-human) by a variety of means. For example, in some embodiments the present invention can be used as a topical cream.

In other embodiments the present invention may be applied orally, as suppository, subcutaneously or topically.

Because the cationic fraction has antimicrobial activity, the present invention can be used to treat microbial conditions that have an inflammatory response, particularly those that lead to excessive inflammatory responses. Thus the present invention could be used to treat such conditions as dandruff (Malassezia furfur), psoriasis (Malassezia furfur and Staphylococcus aureus), eczema (Staphylococcus aureus), Athlete's foot (Trichophyton), and acne (Propionibacterium acnes).

The present invention has a number of advantages over the prior art and these can include the following:
The inclusion of a cationic fraction with an existing anti-inflammatory agent significantly increases the effect of using the agent on its own (i.e potentiating an active ingredient). This can lead to natural treatments being considerably more effective, or possibly the reduction of the synthetic treatments leading to less side-effects, a process known as potentiation.
With a cationic fraction being a “natural” product, this will gain greater consumer acceptance. Further, no discernable side effects have been observed.
The reduction of the fraction from a single step process provides a significant number of useful bioactives as well as decreasing the time labour and costs of extraction process. Thus, a cheaper and more effective formulation can be achieved.
Capturing the advantages of using the whole synergistic cationic fraction over just the inclusion of lactoferrin in an anti-inflammatory application, which may actually act as a pro-inflammatory Alarmin,

The cationic protein fraction can be manufactured in the form of a shelf stable powder suitable for incorporating into capsules, tablets, lozenges, creams, gums, suppositories, bandages, dry films washes, or rinses.

Also, due to the significantly higher bioactivities, smaller doses may be required. For example, typical doses required for chondroitin and glucosamine treatment is on the order of 3-4 g in 2 or 3 large capsules).

A cationic protein fraction from milk has no flavour or odour and may have better efficacy if dose is a chewable formulation so that the active has the opportunity to interact with more of the gut immune system (eg tonsils, Peyer's patches).

### BRIEF DESCRIPTION OF DRAWINGS

Further aspects of the present invention will become apparent from the following description which is given by way of example only and with reference to the accompanying drawings in which:

FIGS. 1 and 2 show dramatically the effect of the present invention compared to the prior art.

### BEST MODES FOR CARRYING OUT THE INVENTION

Below is a trial study showing the effectiveness of the present invention. The term “IDP” mentioned throughout is a treatment composition which includes a cationic fraction as disclosed previously in combination with a cell lysing agent and peroxide generation with approximate percentages as given below.

TABLE 1

| Identity of main protein | M Wt | Isoelectric | |
|---|---|---|---|
| (MS) | kDa | Point | % of total |
| lactoperoxidase¹ | 78 | 8.3 | 15.4% |
| (P80025) | | | |
| quiescin | 63 | 8.69 | 1.3% |
| jacalin-like protein | 13 | 8.71 | 2.0% |
| chitinase-like protein | 43 | 8.74 | 0.5% |
| (P30922) | | | |
| Angiogenin | 16 | 9 | 3.5% |
| (P10152) | | | |
| Lactoferrin | 80 | 8.7 | 77% |
| (P24627) | | | |

¹Lactoperoxidase was determined via extinction coefficient rather than MS

The anti-inflammatory properties of the cationic fraction, IDP, and the combination of IDP with chondroitin and glucosamine were determined by measuring their ability to inhibit the production of the inflammatory compounds, tumor necrosis factor-α (TNF-α) and interleukin-6 (IL-6) produced by LPS activated neutrophils. The efficacy of these test samples was referenced against an un-supplemented control and the effect of the drug chloroquine, a known inhibitor of TNF-α and IL-6 production.

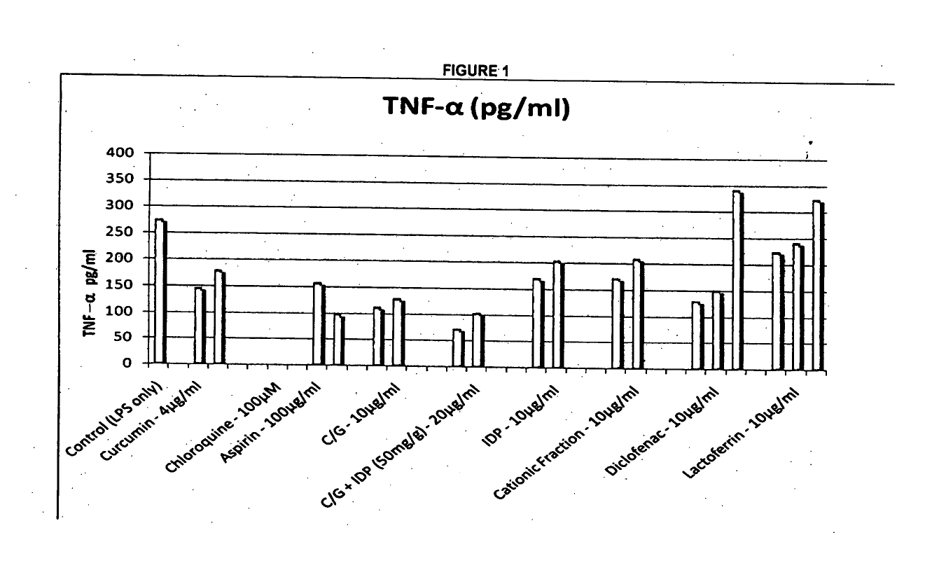

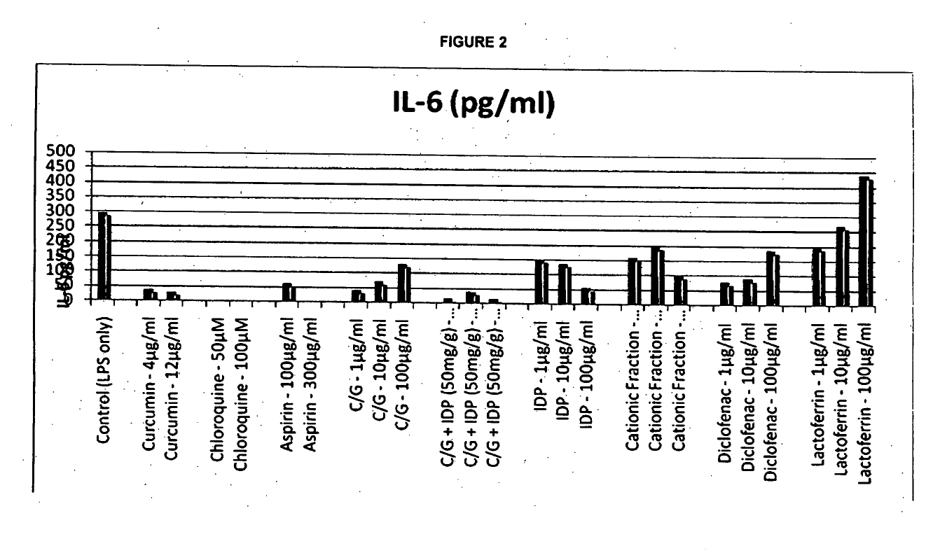

Neutrophils were isolated from rat blood and incubated in the presence of LPS. LPS is derived from bacterial cell walls and is used to mimic a ‘bacterial infection’. This results in the activation of the neutrophils which switch on the production of a variety of cytokines. Two of these cytokine are TNF-α and IL-6. The concentrations of both cytokines produced by neutrophils exposed to LPS and the various test substances are summarised graphically in FIGS. 1 and 2 respectively. The hypothesis of the study was that if the test product had an anti-inflammatory activity it would be able to counter the LPS stimulation and therefore the TNF-α and IL-6 levels observed would be lower than those of the cell control.

Experimental Design

Each sample, the positive controls (Chloroquine, curcumin and aspirin (acetylsalicylic acid) or aspirin derivative) and the negative control (LPS only) were added to cells and cultured in triplicate as described above. Aliquots from each set of triplicates were combined, and duplicates assayed for cytokine concentrations. The plate blank was subtracted from each individual data point.

Measurements

A VersaMax 96-well plate reader was used to colorimetrically assess the production of TNF-α and IL-6 as determined by the ELISA, using Rat TNF-α ELISA kit, R&D Systems, Cat # RTA00, and Rat IL-6 ELISA kit, R&D Systems, Cat # R6000B.

Statistical Methods

The average cytokine concentration, standard deviation and standard error were calculated for each data set (sample and concentration). Preliminary statistical significance was assessed using independent Student t-tests at α≦0.05.

Results and Discussion

i) Summary of Results:
The cell control, which involved stimulation of cells with LPS, showed significant production of both TNF-α and IL-6 (275.38 and 296.84 pg/ml respectively).
The positive control, Chloroquine, had a very strong effect on TNF-α and IL-6 levels, completely inhibiting both TNF-α and IL-6 at both the concentrations tested.
The curcumin sample (a known natural anti-inflammatory) significantly reduced the production of both TNF-α and IL-6. Curcumin reduced TNF-α by approximately 50% while IL-6 was reduced by 90%.
Aspirin yielded the expected results reducing both TNF-α and IL-6 levels significantly.
Diclofenac (Voltaren) was less potent than aspirin or, curcumin and surprisingly displayed an inverse dose response. Diclofenac reduced TNF-α levels by 52% at 1 μg/ml but at 100 μg/ml appeared to stimulate TNF-α. Diclofenac had a stronger inhibitory effect on IL-6 with the inhibition ranging from 37.4% -73.6%.
The nutraceutical (C/G) (chondroitin sulphate:glucosamine sulphate 250 mg:750 mg) also manifested anti-inflammatory properties reducing the levels of both TNF-α and IL-6 by up to 60 and 86% respectively. With respect to both TNF-α and IL-6 levels the C/G mixture displayed an inverse dose response.
When G/C (1 g) was mixed with 50 mg of IDP, there was between 47.9% and 73.5% inhibition of TNF-α production and up to 94.5% inhibition of IL-6 synthesis.
Both the IDP and the cationic fraction demonstrated similar levels of TNF-α inhibition. IDP inhibited TNF-α by up to 38% while the cationic fraction was marginally better at 53%. Both the IDP and the cationic fraction on its own demonstrate significant anti-inflammatory activity with IDP showing stronger inhibition of IL-6 than the cationic fraction. IDP had a much stronger effect on IL-6 inhibiting it by up to 80% while the cationic fraction only inhibited by 67%. The DP and cationic fractions both demonstrated a dose response with the highest concentration of IDP (100 μg/ml) having the strongest anti-IL-6 effect.
At the lowest concentrations tested lactoferrin had no statistically significant effects on either TNF-α or IL-6 production. At the highest concentration tested, lactoferrin stimulated both TNF-α and IL-6 levels by 18 and 48% respectively.

Presentation of Results and Determinations of Statistical Significance:

The data is given as concentration (pg/ml) of TNF-α and IL-6 in Table 2 and FIGS. 1 and 2. Each value is the mean of triplicates±SEM. The level of statistical significance is the effect of each relative to the unsupplemented control.

TABLE 2

| | TNF-α | | | | IL-6 | | | |
|---|---|---|---|---|---|---|---|---|
| | TNF-α | S.E | | % | IL-6 | S.E | | % |
| Sample | (pg/ml) | (pg/ml) | T-test | Inhibition | (pg/ml) | (pg/ml) | T-test | Inhibition |
| Control | 275.38 | 30.81 | 1 | 0% | 296.84 | 7.01 | 1 | 0% |
| (LPS only) | | | | | | | | |
| Curcumin— | 146.71 | 17.64 | 0.068 | 46.7% | 38.65 | 4.08 | 0.001 | 87.0% |
| 4 μg/ml | | | | | | | | |
| Curcumin— | 180.66 | 19.68 | 0.122 | 34.4% | 31.14 | 6.93 | 0.001 | 89.5% |
| 12 μg/ml | | | | | | | | |
| Chloroquine— | 0 | 0 | 0.012 | 100% | 0 | 0 | 0.001 | 100% |
| 50 μM | | | | | | | | |
| Chloroquine— | 0 | 0 | 0.012 | 100% | 0 | 0 | 0.0001 | 100% |
| 100 μM | | | | | | | | |
| Aspirin— | 159.17 | 1.95 | 0.063 | 42.2% | 63.2 | 3.65 | 0.001 | 78.7% |
| 100 μg/ml | | | | | | | | |
| Aspirin— | 99.45 | 12.05 | 0.033 | 63.9% | 0 | 0 | 0.001 | 100% |
| 300 μg/ml | | | | | | | | |
| C/G— | 113.82 | 0.43 | 0.034 | 58.7% | 43.52 | 12.87 | 0.003 | 85.3% |
| 1 μg/ml | | | | | | | | |
| C/G— | 130.7 | 6.03 | 0.044 | 52.5% | 71.23 | 4.37 | 0.001 | 76.0% |
| 10 μg/ml | | | | | | | | |
| C/G— | | | | | 130.22 | 12.91 | 0.008 | 56.1% |
| 100 μg/ml | | | | | | | | |
| C/G + IDP | 72.97 | 1.38 | 0.022 | 73.5% | 16.69 | 3.23 | 0.001 | 94.4% |
| (50 mg/g)— | | | | | | | | |
| 2 μg/ml | | | | | | | | |
| C/G + IDP | 105.16 | 3.28 | 0.031 | 61.8% | 39.52 | 6.56 | 0.001 | 86.7% |
| (50 mg/g)— | | | | | | | | |
| 20 μg/ml | | | | | | | | |
| C/G + IDP | | | | | 16.31 | 16.31 | 0.004 | 94.5% |
| (50 mg/g)— | | | | | | | | |
| 200 μg/ml | | | | | | | | |
| IDP— | 171.55 | 10.05 | 0.085 | 37.7% | 150.72 | 24.67 | 0.029 | 49.2% |
| 1 μg/ml | | | | | | | | |
| IDP— | 204.85 | 0.66 | 0.149 | 25.6% | 137.61 | 20.79 | 0.018 | 53.6% |
| 10 μg/ml | | | | | | | | |
| IDP— | | | | | 57.16 | 6.89 | 0.002 | 80.7% |
| 100 μg/ml | | | | | | | | |
| Cationic | 173.65 | 6.38 | 0.083 | 36.9% | 159.38 | 30.21 | 0.047 | 46.3% |
| Fraction— | | | | | | | | |
| 1 μg/ml | | | | | | | | |
| Cationic | 210.15 | 10.02 | 0.181 | 23.7% | 197.14 | 40.33 | 0.135 | 33.6% |
| Fraction— | | | | | | | | |
| 10 μg/ml | | | | | | | | |
| Cationic | | | | | 100.26 | 15.33 | 0.007 | 66.2% |
| Fraction— | | | | | | | | |
| 100 μg/ml | | | | | | | | |
| Diclofenac— | 132.01 | 5.94 | 0.044 | 52.1% | 78.39 | 18.7 | 0.008 | 73.6% |
| 1 μg/ml | | | | | | | | |
| Diclofenac— | 151.97 | 1.47 | 0.057 | 44.8% | 90.55 | 20.11 | 0.01 | 69.5% |
| 10 μg/ml | | | | | | | | |
| Diclofenac— | 342.46 | 25.72 | 0.236 | −24.4% | 185.71 | 6.96 | 0.008 | 37.4% |
| 100 μg/ml | | | | | | | | |
| Lactoferrin— | 226.19 | 14.7 | 0.286 | 17.9% | 197.88 | 8.95 | 0.0129 | 33.3% |
| 1 μg/ml | | | | | | | | |
| Lactoferrin— | 242.58 | 7.11 | 0.408 | 11.9% | 269.86 | 3.98 | 0.079 | 9.1% |
| 10 μg/ml | | | | | | | | |
| Lactoferrin— | 325.95 | 13.42 | 0.271 | −18.4% | 441.09 | 5.55 | 0.004 | −48.6% |
| 100 μg/ml | | | | | | | | |

Discussion of Results:

The cell control (neutrophils incubated with LPS for 24 hrs) gave excellent results showing the activation of the naïve neutrophils and production of both TNF-α and IL-6 (275.38 and 296.84 pg/ml respectively) (FIGS. 1 and 2).

Curcumin is a compound present in the spice tumeric. There is evidence that it can inhibit TNF-α and IL-6 production by LPS stimulated macrophages (7). Curcumin was tested at two concentrations (4 μg/ml and 12 μg/ml). There was no apparent dose effect. This was the situation for both inflammatory molecules. Curcumin had a stronger effect on IL-6 production and caused close to complete inhibition. This is a somewhat stronger effect than reported elsewhere (7). However the effect on TNF-α synthesis by these concentrations was of the order anticipated.

The drug chloroquine is a known anti-rheumatic and an inhibitor of both TNF-α and IL-6. A recent study has shown that cholorquine prevents the release of TNF-α from macrophages and actually inhibits the formation of IL-6 mRNA (5). Chloroquine was used as the positive control in this study. It completely inhibited both TNF-α and IL-6 at both the concentrations tested and confirming the results obtained by Jang et al (5). The concentrations used in this study were based on those used to produce moderate levels of inhibition in macrophages.

Aspirin yielded the expected results reducing both TNF-α, and IL-6 levels significantly. When tested at 300 μg/ml aspirin was able to reduce the TNF-α level by 64% and completely inhibited the production of IL-6. Aspirin also gave a characteristic dose response with the lower dose of aspirin (100 μg/ml) having a lesser inhibitory effect on both TNF-α and IL-6.

Glucosamine and chondroitin sulphate are natural substances found in and around the cells of cartilage. Glucosamine is distributed in cartilage and other connective tissue, and chondroitin sulphate is a complex carbohydrate that helps cartilage retain water. Glucosamine is also a constituent of glycosaminoglycans (GAGs) including chondroitin sulphate. These supplements are often taken by patients suffering arthritis to ease symptoms and there have been numerous studies to examine the treatment effects of glucosamine and chondroitin (4). This study demonstrated the ability of C/G to reduce the levels of both TNF-α and IL-6 by up to 60 and 86% respectively. It is interesting to note that for both the TNF-α and IL-6 levels the C/G mixture displays an inverse dose response. The actual inhibitory effects for this product for both TNF-α and IL-6 are moderate. At 100 μg/ml the inhibition of TNF-α production was only 31.2% and of IL-6 it was 56.1%. Thus at these concentrations the inhibition was not strong.

IDP had a much stronger effect on IL-6 inhibiting it by up to 80% while the cationic fraction only inhibited by 67%. The IDP showed a direct dose response effect on IL-6. At 1 μg/ml the inhibition was almost 50% which increased marginally at 10 μg/ml. However at 100 μg/ml, IDP inhibited IL-6 production by 80.7%. The cationic fraction was only slightly less active with 1 μg/ml producing 46.3% inhibition, somewhat less at 10 μg/ml (33.6%) but 66.7% at 100 μg/ml.

When the ratio of G/C to IDP is 20:1 the effect on TNF-α production is significant. The 2 μg/ml solution of this combination produced 73.5% inhibition. This solution contains 95 ng/ml of IDP.

So there is a synergistic effect from combining the IDP and the G/C in terms of inhibiting TNF-α production by neutrophils.

A similar effect of the combination is noted with respect to the effect on IL-6 production. When the ratio in the combinations is 20:1 there is strong inhibition of IL-6 production. At 2 μg/ml (equivalent to 95 ng/ml of IDP) the inhibition was 94.4%. So the combination of G/C and IDP is a potent inhibitor of IL-6 production by activated neutrophils. There is a stronger effect than for either of the products alone, especially for IDP, This is similar to that observed when the effect on TNF-α is measured. It would seem that there is a definite synergistic effect.

Voltaren (Diclofenac) was much less potent than aspirin, curcumin or chloroquine and surprisingly it also displayed an inverse dose response curve, with the lower concentration of Voltaren (Diclofenac) (1 μg/ml) producing stronger anti-TNF-α and IL-6 effects than the higher concentration of Voltaren (Diclofenac) (100 μg/ml). In the case of TNF-α, Voltaren (Diclofenac) reduced levels by 52% at 1 μg/ml but at 100 μg/ml it actually appeared to stimulate the production raising the concentration from 275 pg/ml to 342 pg/ml. Voltaren (Diclofenac) appeared to have a stronger inhibitory effect on IL-6 with the inhibitory effect ranging from 74-38%. The primary action is to inhibit cyclo-oxygenase (COX) enzymes. One study reports a reduction in both TNF-α and IL-6 levels following exposure to non-steroidal anti-inflammatory drugs such as diclofenac (8). However other studies have shown Voltaren (Diclofenac) to increase the levels of the inflammatory marker nitric-oxide in astrocytes stimulated with pro-inflammatory cytokines (6) and an in-vivo study suggests that TNF-α levels may increase in patients treated with diclofenac (3).

Overall lactoferrin appeared not to act as anti-inflammatory with respect to both TNF-α and IL-6 production. At the lowest concentrations tested lactoferrin had no statistically significant effect on either TNF-α or IL-6, while at the highest concentration tested lactoferrin appeared to stimulate both TNF-α and IL-6 increasing levels by 18 and 48% respectively. These results may be consistent as lactoferrin is known to act as an alarmin increasing the maturation of human monocytes which in turn up-regulates the production of pro-inflammatory cytokines (1). Surgical patients treated with oral doses of lactoferrin also displayed higher levels of LPS-induced TNF-alpha and IL-6 production compared with patients receiving a placebo (9).

Aspects of the present invention have been described by way of example only and it should be appreciated that modifications and additions may be made thereto without departing from the scope of the appended claims

### REFERENCES

1. de la Rosa G, Yang D, Tewary P, Varadhachary A and Oppenheim J J. Lactoferrin acts as an alarmin to promote the recruitment and activatin of APCs and antigen-specific immune response. J Immnol 2008 May 15; 180 (10): 6868-76

2. Gonzalez E, de la Cruz C, de Nicolas R, Egido J and Herrero-Beaumont G. Long term effect of nosteroidal anti-inflammatory drugs on the production of cytokines and other inflammatory mediators by blood cells of patients with osteoarthritis. Agents Actions. 1994 May; 41 (3-4):171-8

3. Huskisson E C. Glucosamine and chondroitin for osteoarthritis. J Int Med Res. 2008 November-December; 36 (6):1161-79

4. Jang C H, Choi J H, Byun M S and Jue D M. Chloroquine inhibits production of TNF-α, IL-1b and IL-6 from lipopolysaccharide-stimulated human monocytes/macrophages by different modes. Rheumatology. 2006; 45:703-710.

5. Kakita H, Aoyama M. Hussein M H, Kato S, Suzuki S, Ito T, Togari H and Asai K. Diclofenac enhances proinflammatory cytokine-induced nitric oxide production through NF-κβ signalling in cultured astrocytes. Toxicol Appl Pharmacol. 2009 July; 238(1):56-63

6. Liang G, Zhou H, Wang Y, Gurley E C, Feng B, Chen L, Xiao J, Yang S and Li X. Inhibition of LPS-induced production of inflammatory factors in the macrophages by mono-carbonyl analogues of curcumin. J. Cell Mol Med. 2009 Feb. 20.

7. Syggelos S A, Giannopoulou E, Gouvousis P A, Andonopoulos A P, Aletras A J and Panagiotopoulos E. In vitro effects of non-steroidal anti-inflammatory drugs on cytokine, prostanoid and matrix metalloproteinase production by interface membranes from loose hip or knee endoprostheses. Osteoarthritis Cartilage. 2007 May; 15 (5):531-42

8. Zimecki M, Wtaszczyk A, Wojciechowski R, Dawiskiba J and Kruzel M. Lactoferrin regulates the immune response in post-surgical patients. Arch Immunol Ther Exp (Warsz). 2001; 49 (4):325-33

What I/We claim:

1. A medicament including a treatment composition in combination with at least one anti-inflammatory composition,
characterised in that
the treatment composition is extracted from whole milk, processed milk or milk derived substance, and
wherein the treatment composition contains a cationic fraction which includes two or more components of the whole milk, processed milk or milk derived substance with an isoelectric point of or greater than substantially 6.8.

2. A medicament as claimed in claim 1 wherein the anti-inflammatory composition is chloroquine.

3. A medicament as claimed in claim 1 wherein the anti-inflammatory composition is aceticsalicyclic acid or aspirin derivative.

4. A medicament as claimed in claim 1 wherein the anti-inflammatory composition is naturally derived.

5. A medicament as claimed in claim 4 wherein an anti-inflammatory composition is curcumin.

6. A medicament as claimed in either claim 4 or claim 5 wherein an anti-inflammatory composition is glucosomine, chondroitin or a combination thereof.

7. A medicament as claimed in any one of claims 4 to 6 wherein an anti-inflammatory composition is chondroitin.

8. A medicament as claimed in claim 1 wherein an anti-inflammatory composition is an NSAID.

9. A medicament as claimed in claim 1 wherein an anti-inflammatory composition is a corticosteroid.

10. A medicament as claimed in any one of claims 1 to 9 wherein the medicament includes a oxygen and/or peroxide generation system.

11. A medicament as claimed in any one of claims 1 to 10 wherein the medicament includes a cell lysing agent.

12. A medicament as claimed in any one of claims 1 to 11 wherein the treatment composition includes a CLP-1 protein.

13. A medicament as claimed in any one of claims 1 to 12 in the form of a topical cream.

14. A method of preventing or treating inflammatory activity in an animal
characterizing the steps of
a) applying to the animal a medicament as claimed in any one of claims 1 to 13.

15. A method as claimed in claim 14 wherein the inflammatory activity is mastitis.

16. A method as claimed in either claim 14 or 15 wherein the medicament is applied to the animal's mammary gland.

17. The use of a treatment composition in combination with at least one anti-inflammatory composition,
in the manufacture of a medicament for the prevention or treatment of inflammatory activity in an animal,
characterised in that
the treatment composition is extracted from whole milk, processed milk or milk derived substance, and
the treatment composition contains, a cationic fraction which includes 2 or more components of the whole milk, processed milk or milk derived substance with an isoelectric point of or greater than substantially 6.8.

18. A medicament substantially as herein described in the Best Modes Section and illustrated with reference to the accompanying drawings.

19. A method substantially as herein described in the Best Modes Section and illustrated with reference to the accompanying drawings.

20. The use of a treatment substance substantially as herein described in the Best Modes Section and illustrated with reference to the accompanying drawings.
